(19) United States
(12) Patent Application Publication
(10) Pub. No.: US 2021/0207127 A1
(43) Pub. Date: Jul. 8, 2021

(54) LIGANDS AND METHODS OF SELECTING BINDING TARGETS FOR SUCH

(71) Applicant: BICYCLERD, Cambridge (GB)

(72) Inventors: Liuhong CHEN, Cambridge (GB); Christophe BONNY, Cambridge (GB)

(21) Appl. No.: 17/058,722

(22) PCT Filed: May 29, 2019
(86) PCT No.: PCT/EP2019/064094

§ 371 Date: Nov. 25, 2020

(30) Foreign Application Priority Data
May 30, 2018 (GB) 1808835.1

(51) Int. Cl. C12N 15/10; G01N 33/68; G16B 15/30

(52) U.S. Cl. CPC C12N 15/1044; G16B 15/30; G01N 33/6845

(57) ABSTRACT

The present invention provides methods of selecting a target for a ligand, wherein said ligand comprises a polypeptide comprising at least three reactive groups, separated by at least two loop sequences, and a molecular scaffold which forms covalent bonds with the reactive groups of the polypeptide such that at least two polypeptide loops are formed on the molecular scaffold. The present invention also provides said targets, said ligands and methods for using and manufacturing such.

### FIELD

The present invention relates to the field of polypeptide ligands, methods for selecting such ligands and methods of selecting binding targets for such ligands. In particular, the present invention relates to selecting binding targets for polypeptide ligands which comprise at least three reactive groups, separated by at least two loop sequences, and a molecular scaffold which forms covalent bonds with the reactive groups of the polypeptide such that at least two polypeptide loops are formed on the molecular scaffold.

### BACKGROUND

Cyclic peptides are polypeptides in which the amino termini and carboxyl termini; amino termini and side chain; carboxyl termini and side chain; or side chain and side chain are linked with a covalent bond that generates the ring.

Cyclic peptides are able to bind with high affinity and target specificity to protein targets and hence are an attractive molecule class for the development of therapeutics. In fact, several cyclic peptides are successfully used in the clinic, as for example the antibacterial peptide vancomycin, the immunosuppressant drug cyclosporine or the anti-cancer drug ocreotide (Driggers, et al., Nat Rev Drug Discov 2008, 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu, B., et al., Science 330 (6007), 1066-71), a cyclic peptide with the Arg-Gly-Asp motif binding to integrin α∨β3 (355 Å²) (Xiong, J. P., et al., Science 2002, 296 (5565), 151-5) or the cyclic peptide inhibitor upain-1 binding to urokinase-type plasminogen activator (603 Å²; Zhao, G., et al., J Struct Biol 2007, 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, MMP-8) which lost its selectivity over other MMPs when its ring was opened (Cherney, R. J., et al., J Med Chem 1998, 41 (11), 1749-51). The favorable binding properties achieved through macrocyclization are even more pronounced in multicyclic peptides having more than one peptide ring as for example in vancomycin, nisin or actinomycin.

Different research teams have previously tethered polypeptides with cysteine residues to a synthetic molecular structure (Kemp, D. S. and McNamara, P. E., J. Org. Chem, 1985; Timmerman, P. et al., ChemBioChem, 2005). Meloen and co-workers had used tris(bromomethyl)benzene and related molecules for rapid and quantitative cyclisation of multiple peptide loops onto synthetic scaffolds for structural mimicry of protein surfaces (Timmerman, P. et al., ChemBioChem, 2005). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

WO2004/077062 discloses a method of selecting a candidate drug compound. In particular, this document discloses various scaffold molecules comprising first and second reactive groups, and contacting said scaffold with a further molecule to form at least two linkages between the scaffold and the further molecule in a coupling reaction.

WO2006/078161 discloses binding compounds, immunogenic compounds and peptidomimetics. This document discloses the artificial synthesis of various collections of peptides taken from existing proteins. These peptides are then combined with a constant synthetic peptide having some amino acid changes introduced in order to produce combinatorial libraries. By introducing this diversity via the chemical linkage to separate peptides featuring various amino acid changes, an increased opportunity to find the desired binding activity is provided. The constructs disclosed herein typically rely on —SH functionalised peptides, typically comprising cysteine residues, and heteroaromatic groups on the scaffold, typically comprising benzylic halogen substituents such as bis- or tris-bromophenylbenzene. Such groups react to form a thioether linkage between the peptide and the scaffold.

Heinis et al. recently developed a phage display-based combinatorial approach to generate and screen large libraries of bicyclic peptides to targets of interest (Heinis, et al., Nat Chem Biol 2009, 5 (7), 502-7; see also international patent application WO2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene). Bicyclic peptides isolated in selections for affinity to the human proteases cathepsin G and plasma Kallikrein (PK) had nanomolar inhibitory constants. The best inhibitor, PK15, inhibits human PK (hPK) with a Kᵢ of 3 nM. Similarities in the amino acid sequences of several isolated bicyclic peptides suggested that both peptide loops contribute to the binding. PK15 did not inhibit rat PK (81% sequence identity) nor the homologous human serine proteases factor XIa (hfXIa; 69% sequence identity) or thrombin (36% sequence identity) at the highest concentration tested (10 μM) (Heinis, et al., Nat Chem Biol 2009, 5 (7), 502-7). This finding suggests that the bicyclic inhibitor possesses high affinity for its target, and is highly specific.

Improved methods for identifying targets for peptides ligands, in particular for cyclic peptides, is needed in order to identify new uses. In particular, the binding of the ligands to specific targets enables said ligands to be considered as suitable drug-like molecules. The ability to identify a target for a specific ligand quickly, cheaply and/or more efficiently would speed up the drug creation pipeline and enable swifter recognition of suitable molecules for medical uses. Said ligands may also have variable protease stability and solubility profiles which increase their ease of use and suitability. The polypeptide ligands can be used for injection, inhalation, nasal, ocular, oral or topical administration. The use of polypeptide ligands, particularly cyclic peptides or Bicycle® peptides, is further advantageous because they are cheap to produce and easy to use.

### SUMMARY

Accordingly, in one aspect the present invention provides a method of selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising:

(a) screening one or more proteins for the presence of a pocket, said pocket comprising the features of

(i) a volume of about 1000-3000 Å³; and

(ii) at least one solvent-accessible terminus; and

(b) selecting at least one protein which possesses at least one pocket as defined in (a).

Preferably the pocket as defined in (a) also comprises the internal dimensions of (10-30)×(10-30)×(5-30) Å.

Preferably the polypeptide ligand is a cyclic peptide, most preferably a bicyclic peptide.

The solvent accessible surface area of the pocket is preferably at least equivalent to the surface area of a bicycle, that is at least 900-1300 Å².

The solvent-accessible terminus of the pocket is preferably accessible via an opening in the protein at least 5 Å wide.

In one embodiment, the ligand acts as an inhibitor or the target. In a further embodiment, the ligand acts as an agonist of the target. In another embodiment, the ligand has a neutral effect (no change in activity) on the target.

In a second aspect, the present invention provides a method for selecting a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising the steps of:

(a) screening one or more proteins for the presence of a pocket according to the first aspect of the present invention described above, and selecting at least one protein which possesses at least one such pocket; and

(b) contacting said at least one protein with one or more of said ligands, and selecting at least one ligand which binds to said protein.

In a third aspect, the present invention also provides a method for preparing a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising

(a) determining the amino acid sequence of the polypeptide component of a ligand selected according to the methods described in the second aspect above;

(b) synthesising a polypeptide having the sequence determined in (a);

(c) reacting said polypeptide with a molecular scaffold to generate the ligand.

The methods of the present invention may also further comprise the step of determining whether the pocket is located in a protein domain which is involved in a protein-protein interaction with a further protein.

The methods of the present invention may also further comprise the step of exposing the target protein to a library of ligands as defined in claim 1, and selecting one or more ligands which bind to the target protein.

In the methods of the present invention, the molecular scaffold preferably has molecular symmetry corresponding to the number of covalent bonds by which it is attached to the polypeptide. In some embodiments the molecular scaffold possesses threefold molecular symmetry and the molecular scaffold is attached to the polypeptide by three covalent bonds.

In the methods of the present invention, the molecular scaffold may comprise a structurally rigid chemical group. In preferably embodiments the molecular scaffold comprises tris-(bromomethyl)benzene (TBMB), 1,3,5-triacryloyl-1,3,5-triazinane (TATA), N,N′,N″-(benzene-1,3,5-triyl)-tris(2-bromoacetamide) (TBAB) and/or N,N′,N″-benzene-1,3,5-triyltrisprop-2-enamide (TAAB).

In some embodiments of the present invention, the polypeptide comprises a cysteine residue, and wherein at least one of said three covalent bonds for attachment of said molecular scaffold to the polypeptide comprises a bond to said cysteine residue.

In a further aspect, the present invention includes a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, which binds to a target; wherein said target possesses a pocket according to claim 1(a), but is not a polypeptide selected from Kallikrein, MDM2, Cathepsin G.

In one embodiment, said ligand of the present invention comprises a polypeptide comprising at least three reactive groups, separated by at least two loop sequences, and a molecular scaffold which forms covalent bonds with the reactive groups of the polypeptide such that at least two polypeptide loops are formed on the molecular scaffold which binds to a target selected from the group consisting of:

alpha-2-macroglobulin; ATP-binding cassette, sub-family B (MDR/TAP), member 6; ADAM metallopeptidase domain 17; ADAM metallopeptidase domain 33; ADAM metallopeptidase domain 9; adiponectin, C1Q and collagen domain containing; adenosine A3 receptor; adrenoceptor beta 3; agouti related protein homolog (mouse); angiotensin II receptor, type 1; activated leukocyte cell adhesion molecule; apolipoprotein E; apolipoprotein H (beta-2-glycoprotein I); amyloid beta (A4) precursor protein; aquaporin 4; aquaporin 5; beta-site APP-cleaving enzyme 1; bactericidal/permeability-increasing protein; complement component 1, q subcomponent, B chain; complement component 1, q subcomponent, C chain; complement component 1, r subcomponent; complement component 1, s subcomponent; complement component 2; complement component 6; complement component 7; complement component 8, beta polypeptide; carbonic anhydrase XII; carbonic anhydrase IV; carbonic anhydrase VI; CART prepropeptide; cholecystokinin B receptor; chemokine (C-C motif) ligand 11; CD3e molecule, epsilon (CD3-TCR complex); CD3g molecule, gamma (CD3-TCR complex); CD40 molecule, TNF receptor superfamily member 5; CD8a molecule; cytidine deaminase; cadherin 13, H-cadherin (heart); cadherin-related 23; complement factor B; complement factor D (adipsin); complement factor H; chorionic gonadotropin, beta polypeptide; chitinase 3-like 1 (cartilage glycoprotein-39); chitinase, acidic; chitinase 1 (chitotriosidase); chymase 1, mast cell; carnosine dipeptidase 1 (metallopeptidase M20 family); contactin 1; catechol-O-methyltransferase; carboxypeptidase A4; carboxypeptidase B2 (plasma); ceruloplasmin (ferroxidase); carboxypeptidase N, polypeptide 1; complement component (3d/Epstein Barr virus) receptor 2; cathepsin B; cathepsin D; chemokine (C-X-C motif) receptor 4; epidermal growth factor receptor; elastase, neutrophil expressed; EPH receptor A2; v-erb-b2 erythroblastic leukemia viral oncogene homolog 2, neuro/glioblastoma derived oncogene homolog (avian); v-erb-b2 erythroblastic leukemia viral oncogene homolog 3 (avian); v-erb-a erythroblastic leukemia viral oncogene homolog 4 (avian); coagulation factor X; coagulation factor XI; coagulation factor XIII, A1 polypeptide; coagulation factor II (thrombin); coagulation factor II (thrombin) receptor; coagulation factor III (thromboplastin, tissue factor); coagulation factor VII (serum prothrombin conversion accelerator); coagulation factor VIII, procoagulant component; coagulation factor IX; Fc fragment of IgE, low affinity II, receptor for (CD23); Fc fragment of IgG, high affinity Ia, receptor (CD64); Fc fragment of IgG, receptor, transporter, alpha; ficolin (collagen/fibrinogen domain containing lectin) 2 (hucolin); ficolin (collagen/fibrinogen domain containing) 3 (Hakata antigen); folate hydrolase (prostate-specific membrane antigen) 1; follicle stimulating hormone, beta polypeptide; gamma-aminobutyric acid (GABA) B receptor, 2; UDP-N-acetyl-alpha-D-galactosamine:polypeptide N-acetylgalactosaminyltransferase 2 (GalNAc-T2); growth arrest-specific 6; group-specific component (vitamin D binding protein); gamma-glutamyl hydrolase (conjugase, folylpolygammaglutamyl hydrolase); growth hormone 1; growth hormone receptor; ghrelin/obestatin prepropeptide; gastric intrinsic factor (vitamin B synthesis); gastric inhibitory polypeptide receptor; gap junction protein, beta 2, 26 kDa; glycoprotein Ib (platelet), alpha polypeptide; glycoprotein Ib (platelet), beta polypeptide; glycoprotein VI (platelet); glucose-6-phosphate isomerase; glutamate receptor, ionotropic, kainate 1; glutamate receptor, ionotropic, kainate 2; glutamate receptor, metabotropic 1; glutamate receptor, metabotropic 3; glutamate receptor, metabotropic 5; glutamate receptor, metabotropic 7; gelsolin; hemochromatosis; hepatocyte growth factor (hepapoietin A; scatter factor); hedgehog interacting protein; major histocompatibility complex, class I, G; heparan sulfate proteoglycan 2; HtrA serine peptidase 1; hyaluronoglucosaminidase 1; insulin-degrading enzyme; interferon (alpha, beta and omega) receptor 1; interferon (alpha, beta and omega) receptor 2; interferon, gamma; interferon gamma receptor 1; insulin-like growth factor 1 (somatomedin C); insulin-like growth factor 1 receptor; insulin-like growth factor 2 (somatomedin A); insulin-like growth factor 2 receptor; insulin-like growth factor binding protein 1; immunoglobulin heavy constant alpha 1; immunoglobulin heavy constant gamma 1 (G1m marker); immunoglobulin heavy constant gamma 2 (G2m marker); immunoglobulin heavy constant gamma 4 (G4m marker); immunoglobulin heavy constant mu; immunoglobulin kappa constant; immunoglobulin lambda-like polypeptide 1; Indian hedgehog; interleukin 10; interleukin 10 receptor, alpha; interleukin 12A (natural killer cell stimulatory factor 1, cytotoxic lymphocyte maturation factor 1, p35); interleukin 12B (natural killer cell stimulatory factor 2, cytotoxic lymphocyte maturation factor 2, p40); interleukin 17A; interleukin 17F; interleukin 17 receptor A; interleukin 1 receptor, type I; interleukin 1 receptor antagonist; interleukin 21 receptor; interleukin 2 receptor, alpha; interleukin 2 receptor, gamma; interleukin 3 (colony-stimulating factor, multiple); interleukin 4 receptor; interleukin 6 receptor; interleukin 7 receptor; integrin-linked kinase; insulin receptor; itchy E3 ubiquitin protein ligase; integrin, alpha 2b (platelet glycoprotein IIb of IIb/IIIa complex, antigen CD41); integrin, alpha 4 (antigen CD49D, alpha 4 subunit of VLA-4 receptor); integrin, beta 3 (platelet glycoprotein IIIa, antigen CD61); jagged 1; lysyl-tRNA synthetase; kinase insert domain receptor (a type III receptor tyrosine kinase); killer cell immunoglobulin-like receptor, two domains, long cytoplasmic tail, 1; killer cell immunoglobulin-like receptor, two domains, long cytoplasmic tail, 2; killer cell immunoglobulin-like receptor, two domains, long cytoplasmic tail, 3; killer cell immunoglobulin-like receptor, three domains, long cytoplasmic tail, 1; kin of IRRE like 3 (Drosophila); KIT ligand; v-kit Hardy-Zuckerman 4 feline sarcoma viral oncogene homolog; kallikrein-related peptidase 3; KRIT1, ankyrin repeat containing; low density lipoprotein receptor; leptin receptor; leukemia inhibitory factor; leukemia inhibitory factor receptor alpha; lectin, mannose-binding, 1; low density lipoprotein receptor-related protein 6; matrix metallopeptidase 12 (macrophage elastase); matrix metallopeptidase 13 (collagenase 3); matrix metallopeptidase 14 (membrane-inserted); matrix metallopeptidase 1 (interstitial collagenase); matrix metallopeptidase 7 (matrilysin, uterine); matrix metallopeptidase 8 (neutrophil collagenase); myeloperoxidase; neuron navigator 2; natural cytotoxicity triggering receptor 3; neuroligin 4, X-linked; noggin; parathyroid hormone 1 receptor; protein tyrosine phosphatase, receptor type, D; protein tyrosine phosphatase, receptor type, F; poliovirus receptor; renin; ribonuclease, RNase A family, 3; renalase, FAD-dependent amine oxidase; semaphorin 7A, GPI membrane anchor (John Milton Hagen blood group); serpin peptidase inhibitor, clade A (alpha-1 antiproteinase, antitrypsin), member 10; serpin peptidase inhibitor, clade C (antithrombin), member 1; serpin peptidase inhibitor, clade F (alpha-2 antiplasmin, pigment epithelium derived factor), member 1; serpin peptidase inhibitor, clade I (neuroserpin), member 1; superoxide dismutase 3, extracellular; sorbitol dehydrogenase; somatostatin; suppression of tumorigenicity 14 (colon carcinoma); synapsin III; transcobalamin I (vitamin B12 binding protein, R binder family); transcobalamin II; TEK tyrosine kinase, endothelial; transferrin receptor (p90, CD71); transferrin; transforming growth factor, beta 1; transforming growth factor, beta 2; transforming growth factor, beta 3; transforming growth factor, beta receptor 1; transforming growth factor, beta receptor II (70/80 kDa); TIMP metallopeptidase inhibitor 1; TIMP metallopeptidase inhibitor 2; TIMP metallopeptidase inhibitor 3; tolloid-like 1; toll-like receptor 1; toll-like receptor 2; toll-like receptor 3; toll-like receptor 4; toll-like receptor 5; tumor necrosis factor receptor superfamily, member 10b; tumor necrosis factor receptor superfamily, member 13C; tumor necrosis factor receptor superfamily, member 1A; tumor necrosis factor receptor superfamily, member 1B; tumor necrosis factor receptor superfamily, member 4; tumor necrosis factor; tryptase beta 2 (gene/pseudogene); thyroid stimulating hormone receptor; transthyretin; tubby homolog (mouse); tubby like protein 1; vascular cell adhesion molecule 1; vasoactive intestinal peptide receptor 2; pre-B lymphocyte 1; V-set and immunoglobulin domain containing 4; xanthine dehydrogenase; and tyrosyl-tRNA synthetase.

In a further aspect, said ligand is prepared by the method of the third aspect described above.

In a further aspect, the present invention also contemplates the use of a ligand describes above in the treatment of a disease, preferably an inflammatory state, allergic hypersensitivity, cancer, bacterial or viral infection, or an autoimmune disorder.

The present invention also includes a method for identifying a ligand as described above, which is capable of binding to a target, the method comprising
(i) providing a plurality of ligands as described above;
(ii) contacting said plurality of ligands with the target, and
(iii) selecting those ligands which bind said target.

Said method for identifying a ligand may further include the step of determining the sequence of the polypeptide component of said ligand.

Said method for identifying a ligand may further comprise the step of manufacturing a quantity of the ligand isolated as capable of binding to said target. An addition step of manufacturing a quantity of a polypeptide-molecular scaffold conjugate ligand isolated or identified by the method for identifying a ligand as described above may be added. Said manufacture comprises attaching the molecular scaffold to the polypeptide, wherein said polypeptide is recombinantly expressed or chemically synthesized. Said method may further comprise the step of extending the polypeptide at one or more of the N-terminus or the C-terminus of the polypeptide. Said method may further comprise the step of conjugating said polypeptide-molecular scaffold conjugate ligand to a further polypeptide.

The of the conjugation polypeptide-molecular scaffold conjugate ligand to a further polypeptide may be performed by
(i) appending a further cysteine to the polypeptide after bonding to the molecular scaffold, and
(ii) conjugating said polypeptide to said further polypeptide via disulphide bonding to said further cysteine.

A further aspect of the present invention includes computer-implemented method for selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, said method comprising:
(a) interrogating a database of polypeptide structures to identify a protein comprising at least one pocket, said pocket comprising the features of

(i) a volume of about 1000-3000 Å³; and

(ii) at least one solvent-accessible terminus;
(b) identifying, in said database, a first set of proteins comprising at least one pocket as defined in (a);
(c) comparing said first set of proteins with a database of protein domains involved in protein-protein interactions; and
(d) identifying one or more proteins in said first set of proteins which comprise at least one pocket located in a domain putatively responsible for interaction with another protein.

The present invention also contemplates a system for executing the computer-implemented method for selecting a target for a ligand described above.

The present invention also contemplates use of a polypeptide ligand of the invention as a pharmaceutical.

The present invention also contemplates use of a polypeptide ligand of the invention in a method of treatment or diagnosis

### DETAILED DESCRIPTION

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

The present invention relates to methods of selecting targets for polypeptide ligands, said targets, said ligands and methods of use and manufacture of said targets and ligands.

In a most preferred embodiment, the present invention relates to a method of selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising:

(a) screening one or more proteins for the presence of a pocket, said pocket comprising the features of

(i) a volume of about 1000-3000 Å³; and

(ii) at least one solvent-accessible terminus; and

(b) selecting at least one protein which possesses at least one pocket as defined in (a).

### Ligands

As used herein, the term “ligand” refers to an ion or molecule which binds to any molecule, part of a molecule, ion, atom, motif, antibody, epitope, receptor or any part thereof. The ligands as used in present invention preferably comprise or consist of peptides, in most embodiments polypeptides.

A (poly)peptide “ligand” or (poly)peptide “conjugate”, as referred to herein (sometimes referred to herein as simply the “peptide” or “polypeptide” in context), refers to a polypeptide covalently bound to a molecular scaffold. Typically, such polypeptides comprise two or more reactive groups which are capable of forming covalent bonds to a scaffold, and a sequence subtended between said reactive groups which is referred to as the “loop sequence”, since it forms a loop when the peptide is bound to the scaffold. In the present case, the polypeptide ligands comprise at least three reactive groups, and form at least two loops on the scaffold.

The polypeptide ligands of the present invention may be naturally occurring. Preferably the polypeptide ligands of the present invention are synthetic or modified from those found occurring naturally.

The term “protein” as used herein takes its usual meaning in the art and includes derivatised proteins, membrane proteins, cytoskeletal protains and cytoplasmic proteins. A protein comprises any number of amino acids, including naturally occurring amino acids and synthetic amino acids. Any polypeptide also constitutes a protein.

Preferably the polypeptide ligand is a Bicycle®.

In one embodiment the peptide ligand used in the methods of the invention interact with a target pocket having a volume of at least 1000-2500 Å³, more preferably at least 1250-2500 Å³, most preferably at least 1400-2200 Å³.

In one embodiment the peptide ligand used in the method of the invention has a surface area of at least 700-1500 Å², most preferably 900-1300 Å².

The term “reactive groups” as used herein refers groups capable of forming a covalent bond with the molecular scaffold. Typically, the reactive groups are present on amino acid side chains on the peptide ligand. Examples are amino-containing groups such as cysteine, lysine and selenocysteine.

The term “specificity”, in the context herein, refers to the ability of a ligand to bind or otherwise interact with its cognate target to the exclusion of entities which are similar to the target. For example, specificity can refer to the ability of a ligand to inhibit the interaction of a human enzyme, but not a homologous enzyme from a different species. Using the approach described herein, specificity can be modulated, that is increased or decreased, so as to make the ligands more or less able to interact with homologues or paralogues of the intended target. Specificity is not intended to be synonymous with activity, affinity or avidity, and the potency of the action of a ligand on its target (such as, for example, binding affinity or level of inhibition) are not necessarily related to its specificity.

The term “binding activity”, as used herein, refers to quantitative binding measurements taken from binding assays. Therefore, binding activity refers to the amount of peptide ligand which is bound at a given target concentration. Preferred target binding is in the region of 10 to 20 micromolar in primary binding assays. Developed, affinity matured molecules may bind with improved affinity.

Screening for activity, such as binding activity or any other desired activity, is conducted according to methods well known in the art, for instance from phage display technology. For example, targets immobilised to a solid phase can be used to identify and isolate binding members of a repertoire. Screening allows selection of members of a repertoire according to desired characteristics.

Screening a protein for the presence of a pocket can carried out in the first instance by in silico

[symbol]

Multispecificity is the ability to bind to two or more targets. Typically, binding peptides are capable of binding to a single target, such as an epitope in the case of an antibody, due to their conformational properties. However, peptides can be developed which can bind to two or more targets; dual specific antibodies, for example. In the present invention, the peptide ligands can be capable of binding to two or more targets and are therefore be multispecific. Preferably, they bind to two targets, and are dual specific. The binding may be independent, which would mean that the binding sites for the targets on the peptide are not structurally hindered by the binding of one or other of the targets. In this case both targets can be bound independently. More generally it is expected that the binding of one target will at least partially impede the binding of the other.

The term “molecular scaffold” as used herein and further defined below, refers to any molecule which is able to connect a peptide ligand as used in the invention at multiple points to impart one or more structural features to the peptide ligand. It is not a cross-linker, in that it does not merely replace a disulphide bond; instead, it provides two or more attachment points for the peptide. Preferably, the molecular scaffold comprises at least three attachment points for the peptide, referred to as scaffold reactive groups. These groups are capable of reacting to the reactive groups on the peptide to form a covalent bond. Preferred structures for molecular scaffolds are described below

### Targets

Target-ligand complexes show extensive inter- & intra-molecular H-bonding. β-hairpins and γ-turns are common structural motifs in said complexes. At least one solvent-accessible terminus is available, in order to allow the bicycle entry into the pocket for binding.

The target of the current invention binds or otherwise interacts with a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold. Specifically the target binds to other otherwise interacts with a Bicycle® peptide.

Most preferably, the target possesses a pocket as defined below.

Preferably the target is an enzyme, receptor, a growth factor, a complement component, a cell wall component, a hormone, a coagulation factor, an antibody, an epitope, an interleukin, a growth factor, a metallopeptidase, necrosis factor or necrosis factor receptor, or any part thereof.

Most preferably, the target is selected from the group consisting of:

alpha-2-macroglobulin; ATP-binding cassette, sub-family B (MDR/TAP), member 6; ADAM metallopeptidase domain 17; ADAM metallopeptidase domain 33; ADAM metallopeptidase domain 9; adiponectin, C1Q and collagen domain containing; adenosine A3 receptor; adrenoceptor beta 3; agouti related protein homolog (mouse); angiotensin II receptor, type 1; activated leukocyte cell adhesion molecule; apolipoprotein E; apolipoprotein H (beta-2-glycoprotein I); amyloid beta (A4) precursor protein; aquaporin 4; aquaporin 5; beta-site APP-cleaving enzyme 1; bactericidal/permeability-increasing protein; complement component 1, q subcomponent, B chain; complement component 1, q subcomponent, C chain; complement component 1, r subcomponent; complement component 1, s subcomponent; complement component 2; complement component 6; complement component 7; complement component 8, beta polypeptide; carbonic anhydrase XII; carbonic anhydrase IV; carbonic anhydrase VI; CART prepropeptide; cholecystokinin B receptor; chemokine (C-C motif) ligand 11; CD3e molecule, epsilon (CD3-TCR complex); CD3g molecule, gamma (CD3-TCR complex); CD40 molecule, TNF receptor superfamily member 5; CD8a molecule; cytidine deaminase; cadherin 13, H-cadherin (heart); cadherin-related 23; complement factor B; complement factor D (adipsin); complement factor H; chorionic gonadotropin, beta polypeptide; chitinase 3-like 1 (cartilage glycoprotein-39); chitinase, acidic; chitinase 1 (chitotriosidase); chymase 1, mast cell; carnosine dipeptidase 1 (metallopeptidase M20 family); contactin 1; catechol-O-methyltransferase; carboxypeptidase A4; carboxypeptidase B2 (plasma); ceruloplasmin (ferroxidase); carboxypeptidase N, polypeptide 1; complement component (3d/Epstein Barr virus) receptor 2; cathepsin B; cathepsin D; chemokine (C-X-C motif) receptor 4; epidermal growth factor receptor; elastase, neutrophil expressed; EPH receptor A2; v-erb-b2 erythroblastic leukemia viral oncogene homolog 2, neuro/glioblastoma derived oncogene homolog (avian); v-erb-b2 erythroblastic leukemia viral oncogene homolog 3 (avian); v-erb-a erythroblastic leukemia viral oncogene homolog 4 (avian); coagulation factor X; coagulation factor XI; coagulation factor XIII, A1 polypeptide; coagulation factor II (thrombin); coagulation factor II (thrombin) receptor; coagulation factor III (thromboplastin, tissue factor); coagulation factor VII (serum prothrombin conversion accelerator); coagulation factor VIII, procoagulant component; coagulation factor IX; Fc fragment of IgE, low affinity II, receptor for (CD23); Fc fragment of IgG, high affinity Ia, receptor (CD64); Fc fragment of IgG, receptor, transporter, alpha; ficolin (collagen/fibrinogen domain containing lectin) 2 (hucolin); ficolin (collagen/fibrinogen domain containing) 3 (Hakata antigen); folate hydrolase (prostate-specific membrane antigen) 1; follicle stimulating hormone, beta polypeptide; gamma-aminobutyric acid (GABA) B receptor, 2; UDP-N-acetyl-alpha-D-galactosamine:polypeptide N-acetylgalactosaminyltransferase 2 (GalNAc-T2); growth arrest-specific 6; group-specific component (vitamin D binding protein); gamma-glutamyl hydrolase (conjugase, folylpolygammaglutamyl hydrolase); growth hormone 1; growth hormone receptor; ghrelin/obestatin prepropeptide; gastric intrinsic factor (vitamin B synthesis); gastric inhibitory polypeptide receptor; gap junction protein, beta 2, 26 kDa; glycoprotein Ib (platelet), alpha polypeptide; glycoprotein Ib (platelet), beta polypeptide; glycoprotein VI (platelet); glucose-6-phosphate isomerase; glutamate receptor, ionotropic, kainate 1; glutamate receptor, ionotropic, kainate 2; glutamate receptor, metabotropic 1; glutamate receptor, metabotropic 3; glutamate receptor, metabotropic 5; glutamate receptor, metabotropic 7; gelsolin; hemochromatosis; hepatocyte growth factor (hepapoietin A; scatter factor); hedgehog interacting protein; major histocompatibility complex, class I, G; heparan sulfate proteoglycan 2; HtrA serine peptidase 1; hyaluronoglucosaminidase 1; insulin-degrading enzyme; interferon (alpha, beta and omega) receptor 1; interferon (alpha, beta and omega) receptor 2; interferon, gamma; interferon gamma receptor 1; insulin-like growth factor 1 (somatomedin C); insulin-like growth factor 1 receptor; insulin-like growth factor 2 (somatomedin A); insulin-like growth factor 2 receptor; insulin-like growth factor binding protein 1; immunoglobulin heavy constant alpha 1; immunoglobulin heavy constant gamma 1 (G1m marker); immunoglobulin heavy constant gamma 2 (G2m marker); immunoglobulin heavy constant gamma 4 (G4m marker); immunoglobulin heavy constant mu; immunoglobulin kappa constant; immunoglobulin lambda-like polypeptide 1; Indian hedgehog; interleukin 10; interleukin 10 receptor, alpha; interleukin 12A (natural killer cell stimulatory factor 1, cytotoxic lymphocyte maturation factor 1, p35); interleukin 12B (natural killer cell stimulatory factor 2, cytotoxic lymphocyte maturation factor 2, p40); interleukin 17A; interleukin 17F; interleukin 17 receptor A; interleukin 1 receptor, type I; interleukin 1 receptor antagonist; interleukin 21 receptor; interleukin 2 receptor, alpha; interleukin 2 receptor, gamma; interleukin 3 (colony-stimulating factor, multiple); interleukin 4 receptor; interleukin 6 receptor; interleukin 7 receptor; integrin-linked kinase; insulin receptor; itchy E3 ubiquitin protein ligase; integrin, alpha 2b (platelet glycoprotein IIb of IIb/IIIa complex, antigen CD41); integrin, alpha 4 (antigen CD49D, alpha 4 subunit of VLA-4 receptor); integrin, beta 3 (platelet glycoprotein IIIa, antigen CD61); jagged 1; lysyl-tRNA synthetase; kinase insert domain receptor (a type III receptor tyrosine kinase); killer cell immunoglobulin-like receptor, two domains, long cytoplasmic tail, 1; killer cell immunoglobulin-like receptor, two domains, long cytoplasmic tail, 2; killer cell immunoglobulin-like receptor, two domains, long cytoplasmic tail, 3; killer cell immunoglobulin-like receptor, three domains, long cytoplasmic tail, 1; kin of IRRE like 3 (Drosophila); KIT ligand; v-kit Hardy-Zuckerman 4 feline sarcoma viral oncogene homolog; kallikrein-related peptidase 3; KRIT1, ankyrin repeat containing; low density lipoprotein receptor; leptin receptor; leukemia inhibitory factor; leukemia inhibitory factor receptor alpha; lectin, mannose-binding, 1; low density lipoprotein receptor-related protein 6; matrix metallopeptidase 12 (macrophage elastase); matrix metallopeptidase 13 (collagenase 3); matrix metallopeptidase 14 (membrane-inserted); matrix metallopeptidase 1 (interstitial collagenase); matrix metallopeptidase 7 (matrilysin, uterine); matrix metallopeptidase 8 (neutrophil collagenase); myeloperoxidase; neuron navigator 2; natural cytotoxicity triggering receptor 3; neuroligin 4, X-linked; noggin; parathyroid hormone 1 receptor; protein tyrosine phosphatase, receptor type, D; protein tyrosine phosphatase, receptor type, F; poliovirus receptor; renin; ribonuclease, RNase A family, 3; renalase, FAD-dependent amine oxidase; semaphorin 7A, GPI membrane anchor (John Milton Hagen blood group); serpin peptidase inhibitor, clade A (alpha-1 antiproteinase, antitrypsin), member 10; serpin peptidase inhibitor, clade C (antithrombin), member 1; serpin peptidase inhibitor, clade F (alpha-2 antiplasmin, pigment epithelium derived factor), member 1; serpin peptidase inhibitor, clade I (neuroserpin), member 1; superoxide dismutase 3, extracellular; sorbitol dehydrogenase; somatostatin; suppression of tumorigenicity 14 (colon carcinoma); synapsin III; transcobalamin I (vitamin B12 binding protein, R binder family); transcobalamin II; TEK tyrosine kinase, endothelial; transferrin receptor (p90, CD71); transferrin; transforming growth factor, beta 1; transforming growth factor, beta 2; transforming growth factor, beta 3; transforming growth factor, beta receptor 1; transforming growth factor, beta receptor II (70/80 kDa); TIMP metallopeptidase inhibitor 1; TIMP metallopeptidase inhibitor 2; TIMP metallopeptidase inhibitor 3; tolloid-like 1; toll-like receptor 1; toll-like receptor 2; toll-like receptor 3; toll-like receptor 4; toll-like receptor 5; tumor necrosis factor receptor superfamily, member 10b; tumor necrosis factor receptor superfamily, member 13C; tumor necrosis factor receptor superfamily, member 1A; tumor necrosis factor receptor superfamily, member 1B; tumor necrosis factor receptor superfamily, member 4; tumor necrosis factor; tryptase beta 2 (gene/pseudogene); thyroid stimulating hormone receptor; transthyretin; tubby homolog (mouse); tubby like protein 1; vascular cell adhesion molecule 1; vasoactive intestinal peptide receptor 2; pre-B lymphocyte 1; V-set and immunoglobulin domain containing 4; xanthine dehydrogenase; and tyrosyl-tRNA synthetase.

The target may be a whole molecule, group thereof or a part thereof (such as an active site or epitope) of any molecule selected from the above list.

Preferably the peptide ligand interacts with an active site of the target, most preferably binding to said active site.

A target may have more than one binding sites for a polypeptide ligand. The target may bind with one, or more, two or more, three or more, four or more, five or more peptide ligands. Said ligands may be different or the same. The binding of different peptide ligands may have different affects on the target, or may have the same affect. Increasing the number of peptide ligands binding to a target may increase or decrease the effects of the peptide ligand on the target. For example, the agonist or antagonist effect may be increased or decreased.

In one embodiment, binding of the polypeptide ligand to the target has an agonist effect. In another embodiment, said binding has an inhibition or antagonist effect. In another embodiment, said binding has a neutral effect.

### Pockets

As used herein, the term “pocket” refers an indent, depression, hole or space in the three dimensional (secondary, tertiary or quaternary) structure of a target molecule (see Ryan G. Coleman and Kim A. Sharp J Chem lnf Model. 2010 Apr. 26; 50(4): 589-603. doi:10.1021/ci900397t). Said pocket has binding affinity for one or more ligands. Binding may occur via any type of bond or association, such a hydrogen bonding, covalent bonding, ionic bonding or any combination thereof.

The pocket of the current invention comprises specific physical features.

The pocket of the present invention must be large enough to encompass some part of the peptide ligand. Preferably the pocket is large enough to encompass all or most of the peptide ligand. In other words, the peptide ligand can fit at least partially, preferably completely, within the pocket. The ligand becomes at least partially buried in the pocket when binding with the target.

In one embodiment the pocket of the target of the methods of the invention comprises a volume of at least 1000-2500 Å³, more preferably at least 1250-2500 Å³, most preferably at least 1400-2200 Å³.

In one embodiment the pocket of the target of the methods of the invention comprises a surface area of at least 700-1500 Å², most preferably at least 900-1300 Å².

In one embodiment the pocket of the target of the methods of the invention comprises dimensions of at least 30×30×(5 to 10) Å.

In one embodiment the pocket comprises a solvent accessible surface area of at least 900 Å², preferably at least 1000 Å², preferably at least 1300 Å², most preferably at least 1500 Å².

The phrase “solvent accessible surface area” refers to the area of the three dimensional structure of a molecule, such as the secondary, tertiary or quaternary structure of a protein, which can be accessed for contact with a solvent. Preferably said solvent is a liquid solvent, most preferably an aqueous liquid, and preferably such contact leads to binding.

In one embodiment, the pocket comprises at least one solvent-accessible terminus. In some embodiments the ligand comprises at least one solvent accessible terminus.

As noted above, the ligand of the invention, when interacting with the pocket of the invention, preferably becomes at least partially buried within the pocket. Preferably ⅓ to ⅔ of the solvent accessible surface area of the ligand becomes buried in the pocket.

Pockets may be located in various domains of the target. The pocket of the target tends to be biologically relevant. For example, pockets may comprise enzyme active sites or part thereof, binding sites for other or multiple ligands or part thereof, and protein-protein interaction sites or part thereof.

One aspect of the present invention is a method comprising the step of determining whether a pocket is located in a protein domain which is involved in a protein-protein interaction with another protein. This determination may be carried out by any reasonable method in the art, including for example NMR, mass spectroscopy, X-ray crystallography, vibrational spectroscopy, electron microscopy, cryo-electron microscopy or bioinformatics (in silico) methods, or any combination of such methods.

### Other Terms Used in Describing the Methods

The term “contacting” as used herein takes it usual meaning in the art of bringing one molecule into physical contact with another, preferably via a solvent.

Determining the amino acid sequence of any polypeptide component of any ligand, target or other protein used or created in the current invention can be carried out using any suitable technique. Techniques include mass spectrometry, Edman degradation and bioinformatics techniques.

“Synthesising” a polypeptide, protein, amino acid or any other molecule used, identified or created in any aspect of the current invention can be carried out using any suitable technique. Polypeptides may be synthesised biologically. Peptide synthesis is preferably based on Fmoc chemistry, using a Symphony peptide synthesiser manufactured by Peptide Instruments.

The term “reacting” as used here refers to any chemical reaction. Preferably a reaction which forms chemical bonds, preferably hydrogen or covalent bonds.

The term “manufacturing” as used herein refers to making or creating the item to which the term is applied.

### Libraries

In one embodiment, the present invention provides a method comprising the step of exposing the target protein to a library of ligands (preferably as defined in claim 1), and selecting one or more ligands which bind to the target protein

The term “library” as used herein refers to a mixture of heterogeneous polypeptides or nucleic acids. The library is composed of members, which are not identical. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members.

In one embodiment, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

In one embodiment, a library of nucleic acids encodes a repertoire of polypeptides. Each nucleic acid member of the library preferably has a sequence related to one or more other members of the library. By related sequence is meant an amino acid sequence having at least 50% identity, for example at least 60% identity, for example at least 70% identity, for example at least 80% identity, for example at least 90% identity, for example at least 95% identity, for example at least 98% identity, for example at least 99% identity to at least one other member of the library. Identity can be judged across a contiguous segment of at least 3 amino acids, for example at least 4, 5, 6, 7, 8, 9 or 10 amino acids, for example least 12 amino acids, for example least 14 amino acids, for example least 16 amino acids, for example least 17 amino acids or the full length of the reference sequence.

Libraries for use in the present invention may be constructed using techniques known in the art, for example as set forth in WO2004/077062, or biological systems, including phage vector systems as described herein. Other vector systems are known in the art, and include other phage (for instance, phage lambda), bacterial plasmid expression vectors, eukaryotic cell-based expression vectors, including yeast vectors, and the like. For example, see WO2009/098450 or Heinis, et al., Nat Chem Biol 2009, 5 (7), 502-7.

### Molecular Scaffolds

Molecular scaffolds are described in, for example, WO2009/098450 and references cited therein, particularly WO2004077062 and WO2006078161.

As noted above, the term “molecular scaffold” is used herein to refer to any molecule which is able to connect a peptide ligand as used in the invention at multiple points to impart one or more structural features to the peptide ligand.

The molecular scaffold may be a small molecule, such as a small organic molecule.

In one embodiment the molecular scaffold may be, or may be based on, natural monomers such as nucleosides, sugars, or steroids. For example the molecular scaffold may comprise a short polymer of such entities, such as a dimer or a trimer.

In one embodiment the molecular scaffold is a compound of known toxicity, for example of low toxicity. Examples of suitable compounds include cholesterols, nucleotides, steroids, or existing drugs such as tamazepam.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold may comprise or may consist of tris(bromomethyl)benzene, especially 1,3,5-Tris(bromomethyl)benzene (‘TBMB’), or a derivative thereof.

In one embodiment, the molecular scaffold is 2,4,6-Tris(bromomethyl)mesitylene. It is similar to 1,3,5-Tris(bromomethyl)benzene but contains additionally three methyl groups attached to the benzene ring. This has the advantage that the additional methyl groups may form further contacts with the polypeptide and hence add additional structural constraint.

Other molecular scaffolds include 1,3,5-triacryloyl-1,3,5-triazinane (TATA), N,N′,N″-(benzene-1,3,5-triyl)-tris(2-bromoacetamide) (TBAB) and N,N′,N″-benzene-1,3,5-triyltrisprop-2-enamide (TAAB). See Chen et al., ChemBioChem 2012, 13, 1032-1038. Preferably the molecular scaffold comprises a structurally rigid chemical group.

The molecular scaffold used in the methods of the invention contains chemical groups that allow functional groups of the polypeptide of the ligand used in the methods of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

The molecular scaffold preferably has molecular symmetry corresponding to the number of covalent bonds by which it is attached to the polypeptide. Preferably the molecular scaffold possesses threefold molecular symmetry and the molecular scaffold is attached to the polypeptide by three covalent bonds.

### Computer Implementation

The present invention includes computer-implemented methods. Preferably a computer-implemented for selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, said method comprising:
(a) interrogating a database of polypeptide structures to identify a protein comprising at least one pocket, said pocket comprising the features of

(i) a volume of about 1000-3000 Å³; and

(ii) at least one solvent-accessible terminus;
(b) identifying, in said database, a first set of proteins comprising at least one pocket as defined in (a);
(c) comparing said first set of proteins with a database of protein domains involved in protein-protein interactions; and
(d) identifying one or more proteins in said first set of proteins which comprise at least one pocket located in a domain putatively responsible for interaction with another protein.

Other methods of the invention may also be computer implemented.

### Medical Uses

The methods of the present invention may be used to treat, prevent, suppress and/or ameliorate disease, disease symptoms and medical conditions, and also in diagnosis.

The peptide ligands used the present invention will typically find use in preventing, suppressing or treating inflammatory states, allergic hypersensitivity, cancer, bacterial or viral infection, and autoimmune disorders (which include, but are not limited to, Type I diabetes, psoriasis, multiple sclerosis, rheumatoid arthritis, systemic lupus erythematosus, Crohn's disease and myasthenia gravis). Using the methods of the present invention to identify targets for such ligands also assists in identifying which diseases a ligand may be used to treat, prevent and/or ameliorate.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

After carrying out the method of selecting a target for a ligand of the invention, or the method for selecting a ligand, a further step of treating, ameliorating, or improving the symptoms of a medical condition. Preferably this may be carried out by treating a human or animal patient with the ligand. The ligands of the invention may be used as drugs or drug-like molecules. The ligand may be formulated for injection, inhalation, nasal, ocular, oral or topical administration.

The present invention therefore includes a method of selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising:

(a) screening one or more proteins for the presence of a pocket, said pocket comprising the features of

(i) a volume of about 1000-3000 Å³; and

(ii) at least one solvent-accessible terminus;

(b) selecting at least one protein which possesses at least one pocket as defined in (a); and

(c) using the ligand to bind to or interact with the target in a method of medical treatment or diagnosis.

The methods of the present invention are further discussed below. Said methods are preferably carried out in vitro. Methods of the invention may be carried out in vivo.

Methods of the invention may be carried out on a sample taken from a human or animal patient. Such a sample may be for example blood, mucus, skin or plasma. Such methods are preferably methods of diagnosis and preferably carried out in vitro and not practiced directly on the human or animal body.

The present invention also contemplates the use of a ligand, selected by a method of the invention, in a pharmaceutical.

Generally, the peptide ligands will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cylcosporine, methotrexate, adriamycin or cisplatinum, and immunotoxins. Pharmaceutical compositions can include “cocktails” of various cytotoxic or other agents in conjunction with the selected antibodies, receptors or binding proteins thereof of the present invention, or even combinations of selected polypeptides according to the present invention having different specificities, such as polypeptides selected using different target ligands, whether or not they are pooled prior to administration.

The present invention also contemplates use of a ligand of the invention in the manufacture of a medicament for the treatment, prevention, suppression and/or amelioration of disease, disease symptoms and medical conditions.

### Methods

The methods of the present invention may be carried out using any techniques and/or equipment known in the art. Preferably said methods are carried out using laboratory techniques.

The first method of the invention is a method of selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising:

(a) screening one or more proteins for the presence of a pocket, said pocket comprising the features of

(i) a volume of about 1000-3000 Å³; and

(ii) at least one solvent-accessible terminus;

(b) selecting at least one protein which possesses at least one pocket as defined in (a).

Also herein is contemplated a method for selecting a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising the steps of:

(a) screening one or more proteins for the presence of a pocket according to claim 1 or claim 2, and selecting at least one protein which possesses at least one such pocket; and

(b) contacting said at least one protein with one or more of said ligands, and selecting at least one ligand which binds to said protein.

One embodiment of the present invention comprises a method for preparing a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising

(a) determining the amino acid sequence of the polypeptide component of a ligand selected according to one of the methods described above;

(b) synthesising a polypeptide having the sequence determined in (a);

(c) reacting said polypeptide with a molecular scaffold to generate the ligand.

A method for identifying a ligand according to any of the above-described methods, wherein said ligand is capable of binding to a target, is also contemplated. The method comprising
(i) providing a plurality of ligands according to any of the previous methods;
(ii) contacting said plurality of ligands with the target, and
(iii) selecting those ligands which bind said target.

Said method may further comprise the step of manufacturing a quantity of a polypeptide-molecular scaffold conjugate ligand isolated or identified by the method, preferably wherein said manufacture comprises attaching the molecular scaffold to the polypeptide, preferably wherein said polypeptide is recombinantly expressed or chemically synthesized.

Said method of identifying a ligand may further comprise the step of extending the polypeptide at one or more of the N-terminus or the C-terminus of the polypeptide.

Said method of identifying a ligand may further comprise the step of conjugating said polypeptide-molecular scaffold conjugate ligand to a further polypeptide. Preferably said conjugation is performed by
(i) appending a further cysteine to the polypeptide after bonding to the molecular scaffold, and
(ii) conjugating said polypeptide to said further polypeptide via disulphide bonding to said further cysteine.

The methods of the invention are preferably carried out in vitro.

Methods of the invention may be carried out on any sample. Preferably said sample is a liquid sample. Methods of the invention may also be used to select a target from a library (as described above).

Methods of the invention, or some steps of said methods, may be computer implemented.

Various modifications and variations of the described methods and system of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

The present invention is further illustrated with the following examples.

### REFERENCES

Driggers, et al., Nat Rev Drug Discov 2008, 7 (7), 608-24
Wu, B., et al., Science 330 (6007), 1066-71
Xiong, J. P., et al., Science 2002, 296 (5565), 151-5
Zhao, G., et al., J Struct Biol 2007, 160 (1), 1-10
Cherney, R. J., et al., J Med Chem 1998, 41 (11), 1749-51
Kemp, D. S. and McNamara, P. E., J. Org. Chem, 1985
Timmerman, P. et al., ChemBioChem, 2005
WO 2004/077062
WO 2006/078161
Heinis, et al., Nat Chem Biol 2009, 5 (7), 502-7
WO2009/098450
Mack (1982) Remington's Pharmaceutical Sciences, 16th Edition
Chen et al., ChemBioChem 2012, 13, 1032-1038

1. A method of selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising:
(a) screening one or more proteins for the presence of a pocket, said pocket comprising the features of
(i) a volume of about 1000-3000 Å³;
(ii) at least one solvent-accessible terminus; and
(b) selecting at least one protein which possesses at least one pocket as defined in (a).

2. A method of claim 1 wherein the pocket as defined in (a) further comprises the internal dimensions of (10-30)×(10-30)×(5-30) Å.

3. A method for selecting a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising the steps of:
(a) screening one or more proteins for the presence of a pocket according to claim 1 or claim 2, and selecting at least one protein which possesses at least one such pocket; and
(b) contacting said at least one protein with one or more of said ligands, and selecting at least one ligand which binds to said protein.

4. A method for preparing a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, comprising
(a) determining the amino acid sequence of the polypeptide component of a ligand selected according to claim 2;
(b) synthesising a polypeptide having the sequence determined in (a);
(c) reacting said polypeptide with a molecular scaffold to generate the ligand.

5. A method according to any preceding claim, further comprising determining whether the pocket is located in a protein domain which is involved in a protein-protein interaction with a further protein.

6. A method according to any preceding claim, further comprising exposing the target protein to a library of ligands as defined in claim 1, and selecting one or more ligands which bind to the target protein.

7. A method according to any preceding claim wherein the molecular scaffold has molecular symmetry corresponding to the number of covalent bonds by which it is attached to the polypeptide.

8. A method according to claim 7 wherein the molecular scaffold possesses threefold molecular symmetry and the molecular scaffold is attached to the polypeptide by three covalent bonds.

9. A method according to any preceding claim wherein the molecular scaffold comprises a structurally rigid chemical group.

10. A method according to claim 9 wherein the molecular scaffold comprises tris-(bromomethyl)benzene (TBMB), 1,3,5-triacryloyl-1,3,5-triazinane (TATA), N,N′,N″-(benzene-1,3,5-triyl)-tris(2-bromoacetamide) (TBAB) and/or N,N′,N″-benzene-1,3,5-triyltrisprop-2-enamide (TAAB).

11. A method according to any preceding claim wherein said polypeptide comprises a cysteine residue, and wherein at least one of said three covalent bonds for attachment of said molecular scaffold to the polypeptide comprises a bond to said cysteine residue.

12. A ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, which binds to a target; wherein said target possesses a pocket according to claim 1(a), but is not a polypeptide selected from Kallikrein, MDM2, Cathepsin G.

13. A ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold which binds to a target selected from the group consisting of:
alpha-2-macroglobulin; ATP-binding cassette, sub-family B (MDR/TAP), member 6; ADAM metallopeptidase domain 17; ADAM metallopeptidase domain 33; ADAM metallopeptidase domain 9; adiponectin, C1Q and collagen domain containing; adenosine A3 receptor; adrenoceptor beta 3; agouti related protein homolog (mouse); angiotensin II receptor, type 1; activated leukocyte cell adhesion molecule; apolipoprotein E; apolipoprotein H (beta-2-glycoprotein I); amyloid beta (A4) precursor protein; aquaporin 4; aquaporin 5; beta-site APP-cleaving enzyme 1; bactericidal/permeability-increasing protein; complement component 1, q subcomponent, B chain; complement component 1, q subcomponent, C chain; complement component 1, r subcomponent; complement component 1, s subcomponent; complement component 2; complement component 6; complement component 7; complement component 8, beta polypeptide; carbonic anhydrase XII; carbonic anhydrase IV; carbonic anhydrase VI; CART prepropeptide; cholecystokinin B receptor; chemokine (C-C motif) ligand 11; CD3e molecule, epsilon (CD3-TCR complex); CD3g molecule, gamma (CD3-TCR complex); CD40 molecule, TNF receptor superfamily member 5; CD8a molecule; cytidine deaminase; cadherin 13, H-cadherin (heart); cadherin-related 23; complement factor B; complement factor D (adipsin); complement factor H; chorionic gonadotropin, beta polypeptide; chitinase 3-like 1 (cartilage glycoprotein-39); chitinase, acidic; chitinase 1 (chitotriosidase); chymase 1, mast cell; carnosine dipeptidase 1 (metallopeptidase M20 family); contactin 1; catechol-O-methyltransferase; carboxypeptidase A4; carboxypeptidase B2 (plasma); ceruloplasmin (ferroxidase); carboxypeptidase N, polypeptide 1; complement component (3d/Epstein Barr virus) receptor 2; cathepsin B; cathepsin D; chemokine (C-X-C motif) receptor 4; epidermal growth factor receptor; elastase, neutrophil expressed; EPH receptor A2; v-erb-b2 erythroblastic leukemia viral oncogene homolog 2, neuro/glioblastoma derived oncogene homolog (avian); v-erb-b2 erythroblastic leukemia viral oncogene homolog 3 (avian); v-erb-a erythroblastic leukemia viral oncogene homolog 4 (avian); coagulation factor X; coagulation factor XI; coagulation factor XIII, A1 polypeptide; coagulation factor II (thrombin); coagulation factor II (thrombin) receptor; coagulation factor III (thromboplastin, tissue factor); coagulation factor VII (serum prothrombin conversion accelerator); coagulation factor VIII, procoagulant component; coagulation factor IX; Fc fragment of IgE, low affinity II, receptor for (CD23); Fc fragment of IgG, high affinity Ia, receptor (CD64); Fc fragment of IgG, receptor, transporter, alpha; ficolin (collagen/fibrinogen domain containing lectin) 2 (hucolin); ficolin (collagen/fibrinogen domain containing) 3 (Hakata antigen); folate hydrolase (prostate-specific membrane antigen) 1; follicle stimulating hormone, beta polypeptide; gamma-aminobutyric acid (GABA) B receptor, 2; UDP-N-acetyl-alpha-D-galactosamine:polypeptide N-acetylgalactosaminyltransferase 2 (GalNAc-T2); growth arrest-specific 6; group-specific component (vitamin D binding protein); gamma-glutamyl hydrolase (conjugase, folylpolygammaglutamyl hydrolase); growth hormone 1; growth hormone receptor; ghrelin/obestatin prepropeptide; gastric intrinsic factor (vitamin B synthesis); gastric inhibitory polypeptide receptor; gap junction protein, beta 2, 26 kDa; glycoprotein Ib (platelet), alpha polypeptide; glycoprotein Ib (platelet), beta polypeptide; glycoprotein VI (platelet); glucose-6-phosphate isomerase; glutamate receptor, ionotropic, kainate 1; glutamate receptor, ionotropic, kainate 2; glutamate receptor, metabotropic 1; glutamate receptor, metabotropic 3; glutamate receptor, metabotropic 5; glutamate receptor, metabotropic 7; gelsolin; hemochromatosis; hepatocyte growth factor (hepapoietin A; scatter factor); hedgehog interacting protein; major histocompatibility complex, class I, G; heparan sulfate proteoglycan 2; HtrA serine peptidase 1; hyaluronoglucosaminidase 1; insulin-degrading enzyme; interferon (alpha, beta and omega) receptor 1; interferon (alpha, beta and omega) receptor 2; interferon, gamma; interferon gamma receptor 1; insulin-like growth factor 1 (somatomedin C); insulin-like growth factor 1 receptor; insulin-like growth factor 2 (somatomedin A); insulin-like growth factor 2 receptor; insulin-like growth factor binding protein 1; immunoglobulin heavy constant alpha 1; immunoglobulin heavy constant gamma 1 (G1m marker); immunoglobulin heavy constant gamma 2 (G2m marker); immunoglobulin heavy constant gamma 4 (G4m marker); immunoglobulin heavy constant mu; immunoglobulin kappa constant; immunoglobulin lambda-like polypeptide 1; Indian hedgehog; interleukin 10; interleukin 10 receptor, alpha; interleukin 12A (natural killer cell stimulatory factor 1, cytotoxic lymphocyte maturation factor 1, p35); interleukin 12B (natural killer cell stimulatory factor 2, cytotoxic lymphocyte maturation factor 2, p40); interleukin 17A; interleukin 17F; interleukin 17 receptor A; interleukin 1 receptor, type I; interleukin 1 receptor antagonist; interleukin 21 receptor; interleukin 2 receptor, alpha; interleukin 2 receptor, gamma; interleukin 3 (colony-stimulating factor, multiple); interleukin 4 receptor; interleukin 6 receptor; interleukin 7 receptor; integrin-linked kinase; insulin receptor; itchy E3 ubiquitin protein ligase; integrin, alpha 2b (platelet glycoprotein IIb of IIb/IIIa complex, antigen CD41); integrin, alpha 4 (antigen CD49D, alpha 4 subunit of VLA-4 receptor); integrin, beta 3 (platelet glycoprotein IIIa, antigen CD61); jagged 1; lysyl-tRNA synthetase; kinase insert domain receptor (a type III receptor tyrosine kinase); killer cell immunoglobulin-like receptor, two domains, long cytoplasmic tail, 1; killer cell immunoglobulin-like receptor, two domains, long cytoplasmic tail, 2; killer cell immunoglobulin-like receptor, two domains, long cytoplasmic tail, 3; killer cell immunoglobulin-like receptor, three domains, long cytoplasmic tail, 1; kin of IRRE like 3 (Drosophila); KIT ligand; v-kit Hardy-Zuckerman 4 feline sarcoma viral oncogene homolog; kallikrein-related peptidase 3; KRIT1, ankyrin repeat containing; low density lipoprotein receptor; leptin receptor; leukemia inhibitory factor; leukemia inhibitory factor receptor alpha; lectin, mannose-binding, 1; low density lipoprotein receptor-related protein 6; matrix metallopeptidase 12 (macrophage elastase); matrix metallopeptidase 13 (collagenase 3); matrix metallopeptidase 14 (membrane-inserted); matrix metallopeptidase 1 (interstitial collagenase); matrix metallopeptidase 7 (matrilysin, uterine); matrix metallopeptidase 8 (neutrophil collagenase); myeloperoxidase; neuron navigator 2; natural cytotoxicity triggering receptor 3; neuroligin 4, X-linked; noggin; parathyroid hormone 1 receptor; protein tyrosine phosphatase, receptor type, D; protein tyrosine phosphatase, receptor type, F; poliovirus receptor; renin; ribonuclease, RNase A family, 3; renalase, FAD-dependent amine oxidase; semaphorin 7A, GPI membrane anchor (John Milton Hagen blood group); serpin peptidase inhibitor, clade A (alpha-1 antiproteinase, antitrypsin), member 10; serpin peptidase inhibitor, clade C (antithrombin), member 1; serpin peptidase inhibitor, clade F (alpha-2 antiplasmin, pigment epithelium derived factor), member 1; serpin peptidase inhibitor, clade I (neuroserpin), member 1; superoxide dismutase 3, extracellular; sorbitol dehydrogenase; somatostatin; suppression of tumorigenicity 14 (colon carcinoma); synapsin III; transcobalamin I (vitamin B12 binding protein, R binder family); transcobalamin II; TEK tyrosine kinase, endothelial; transferrin receptor (p90, CD71); transferrin; transforming growth factor, beta 1; transforming growth factor, beta 2; transforming growth factor, beta 3; transforming growth factor, beta receptor 1; transforming growth factor, beta receptor II (70/80 kDa); TIMP metallopeptidase inhibitor 1; TIMP metallopeptidase inhibitor 2; TIMP metallopeptidase inhibitor 3; tolloid-like 1; toll-like receptor 1; toll-like receptor 2; toll-like receptor 3; toll-like receptor 4; toll-like receptor 5; tumor necrosis factor receptor superfamily, member 10b; tumor necrosis factor receptor superfamily, member 13C; tumor necrosis factor receptor superfamily, member 1A; tumor necrosis factor receptor superfamily, member 1B; tumor necrosis factor receptor superfamily, member 4; tumor necrosis factor; tryptase beta 2 (gene/pseudogene); thyroid stimulating hormone receptor; transthyretin; tubby homolog (mouse); tubby like protein 1; vascular cell adhesion molecule 1; vasoactive intestinal peptide receptor 2; pre-B lymphocyte 1; V-set and immunoglobulin domain containing 4; xanthine dehydrogenase; and tyrosyl-tRNA synthetase.

14. A ligand according to claim 12 or claim 13, when prepared by the method of claim 3.

15. Use of a ligand according to any one of claims 12-14 in the treatment of a disease, preferably an inflammatory state, allergic hypersensitivity, cancer, bacterial or viral infection, or an autoimmune disorder.

16. A method for identifying a ligand according to any one of claims 12 to 15 which is capable of binding to a target, the method comprising
(i) providing a plurality of ligands according to any one of claims 12 to 15;
(ii) contacting said plurality of ligands with the target, and
(iii) selecting those ligands which bind said target.

17. A method according to claim 16 further comprising determining the sequence of the polypeptide component of said ligand.

18. A method according to claim 16 or claim 17 further comprising the step of manufacturing a quantity of the ligand isolated as capable of binding to said target.

19. A method according to claim 18 further comprising the step of manufacturing a quantity of a polypeptide-molecular scaffold conjugate ligand isolated or identified by the method claim 14 or claim 15, said manufacture comprising attaching the molecular scaffold to the polypeptide, wherein said polypeptide is recombinantly expressed or chemically synthesized.

20. A method according to claim 19 further comprising the step of extending the polypeptide at one or more of the N-terminus or the C-terminus of the polypeptide.

21. A method according to any of claim 19 or 20 further comprising the step of conjugating said polypeptide-molecular scaffold conjugate ligand to a further polypeptide.

22. A method according to claim 20 wherein said conjugation is performed by
(i) appending a further cysteine to the polypeptide after bonding to the molecular scaffold, and
(ii) conjugating said polypeptide to said further polypeptide via disulphide bonding to said further cysteine.

23. A computer-implemented method for selecting a target for a ligand comprising a polypeptide comprising at least three reactive groups, separated by at least two loop sequences, and a molecular scaffold which forms covalent bonds with the reactive groups of the polypeptide such that at least two polypeptide loops are formed on the molecular scaffold, said method comprising:
(a) interrogating a database of polypeptide structures to identify a protein comprising at least one pocket, said pocket being defined by
(i) a volume of about 1000-3000 Å³; and
(ii) at least one solvent-accessible terminus; and
(b) identifying, in said database, a first set of proteins comprising at least one pocket as defined in (a);
(c) comparing said first set of proteins with a database of protein domains involved in protein-protein interactions; and
(d) identifying one or more proteins in said first set of proteins which comprise at least one pocket located in a domain putatively responsible for interaction with another protein.
